(19) United States
(12) Patent Application Publication
(10) Pub. No.: US 2025/0161327 A1
(43) Pub. Date: May 22, 2025

(54) PREPARATION OF NOVEL TRITERPENE ALCOHOL DERIVATIVES WITH ENHANCED BIOAVAILABILITY FOR CANCER, INFLAMMATION AND PAIN TREATMENT

(71) Applicant: LivFul Inc., Alpharetta, GA (US)

(72) Inventor: Luiz Francisco PIANOWSKI, ITATIBA (BR)

(21) Appl. No.: 18/836,613

(22) PCT Filed: Feb. 7, 2023
(86) PCT No.: PCT/BR2023/050041

§ 371 Date: Aug. 7, 2024

Related U.S. Application Data
(60) Provisional application No. 63/307,348, filed on Feb. 7, 2022.

(51) Int. Cl. A61K 31/575; A61K 38/45

(52) U.S. Cl. CPC A61K 31/575; A61K 38/45; C12Y 207/11013

(57) ABSTRACT

The invention generally refers to pharmaceutical uses of tetracyclic terpene 3-ols, as an example lanosta-8,24-dien-3-ol, bearing polar and/or charged moieties, as anti-inflammatory, anti-cancer and analgesic agents via the inhibition of the disordered activation of serine-threonine protein kinases, particularly PKC.

### FIELD OF THE INVENTION

The invention generally refers to pharmaceutical uses of tetracyclic terpene 3-ols, as an example lanosta-8,24-dien-3-ol, bearing polar and/or charged moieties, as anti-inflammatory, anti-cancer and analgesic agents via the inhibition of the disordered activation of serine-threonine protein kinases, particularly PKC.

### STATE OF THE ART

Natural compounds like tetracyclic triterpenes attracted more attention due to their anti-cancer and anti-inflammatory activities. However, the pharmacokinetics studies show their poor oral bioavailability associated with their poor solubility in aqueous media and permeability. Their formulations are a hit-or-miss and highly dependent on used excipients and processes, increasing overall their production cost. It is essential to optimize the parent compound structure to make it more water-soluble.

### DESCRIPTION OF INVENTION

Triterpene alcohols are virtually insoluble in water. We considered polar or charged moieties to be introduced onto the selected triterpene alcohol group. Such derivatives bearing polar, negatively or positively charged groups will have the ability of mixing with water and lipids as well, like any detergent molecule. The preferred modifying moieties are chosen from these with a transient stability, which can be affected by tissue environment and/or local enzymatic activities. The proposed modifications R strive to preserve the core triterpene structure intact, as shown below.

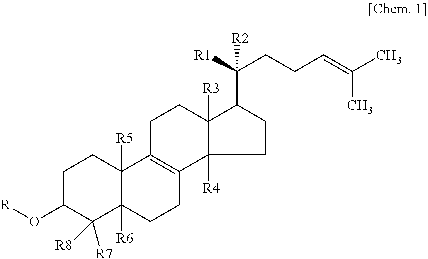

Wherein R—preferred polar or charged moiety, R1, R2, R3, R4, R5, R6, R7, R8 moieties as hydrogen, hydroxyl, methyl group, hydroxymethyl, carboxyl or their combination.

1. Triterpene sulfates. Triterpene sulfates occur as metabolites in some species. Sulfate esters of steroids posses are known for better solubility in water than steroids themselves. The triterpene alcohol sulfate esters are prepared using chlorosulfonic acid in basic media. The sulfate can be converted further into sodium, potassium or lithium salts. Lithium salts are considered for their superior solubility in water, better than salts with other metals.

2. Triterpene phosphates. Phosphate ester derivative is a very appealing candidate as a charged modifier. The esters can be synthesized using phosphoryl chloride, or alternatively using one of the common phosphorylating reagents employed in nucleic acids chemistry. The triterpene alcohol phosphate ester can be further formulated into salts with metal ions or organic bases.

3. Triterpene conjugates with amino acids. To expand the versatility of polar modifying moieties, we considered syntheses of conjugates with amino acids, which can be predominantly acidic, basic or polar in nature. Lysine and arginine are especially attractive for that purpose. Further, the ideas can be extended to peptides with an affinity to specific receptors, as the amino acids form convenient handles for further modifications.

4. Triterpene conjugated with PEG polymers. PEG technology was applied successfully in drug formulation to improve drugs bioavailability. PEG polymers have a natural tendency to wrap around drug molecules forming a polar surface. PEG carboxylic acids with molecular weight from 500 to 4,000 daltons are sufficient to achieve this objective. It is also uncommon to employ larger PEG polymers than 4,000 daltons. PEG conjugates are known to slowly decompose in vivo releasing the core compounds.

6. Triterpene conjugated with carbohydrates. The carbohydrates can improve solubility of selected triterpenes, and also can be employed as guiding molecule utilizing cellular transport mechanisms. The utility of the modification might rely predominantly on latter than the solubility in aqucous media.

7. Encapsulation of triterpenes in complexes with cyclodextrins. This approach is based on modifiers without covalent bonding to triterpene. Known examples are ginsenosides, biologically active triterpenes in ginseng, which were successfully loaded into beta-cyclodextrins.

8. Derivatization of selected triterpenes with dicarboxylic acids. Glutaric and succinic acids can be easily employed. The transformation of triterpene alcohol group into carboxylic may increase its solubility or serve as a convenient handle for introduction of more polar moieties.

In an embodiment, cuphol succinate was active in the cell code (MDA-MB-231). The inhibitory activity began with doses less than 30 ug/ml, so much so that IC50 was 17 ug/ml. This is a triple negative breast cancer cell.

According to the present invention, it was verified an inhibition of cytokines, supporting the treatment of COVID-19.

### EXAMPLES

### Cell viability assay

Evaluation of the viability of MDA-MB-231 cells after incubation with Eu-succ through the MTT assay. The cells were incubated with the Test Item (Eu-succ: 0.01-100 μg/mL) for 24 hours, and then the cell viability assay was performed through the MTT method. The vertical bars represent the mean±standard error of the mean of 3 different trials in triplicate. The test item presented IC50: 17.22 μg/mL (10.53-27.28 μg/mL). The viability percentage was calculated in relation to the Vehicle group (RPMI1640 culture medium with 1% DMSO).

### Feasibility Test—MTT

Evaluation of the viability of A549, Caco-2, MDA-MB-231 and THP-1 cells after incubation with the test item ST-160.1 and ST-160.2 through the MTT assay. The cells were incubated with the Test Items (ST-160.1 and ST-160.2; 1-30 μg/mL) for 24 hours, and then the cell viability assay was performed through the MTT method. An essay was made in duplicate. (A) ST-160.1 and em (B) ST-160.2. The viability percentage was calculated in relation to the Vehicle group (DMEM or RPMI1640 culture medium with 1% DMSO).

Evaluation of the viability of A549, Caco-2, MDA-MB-231 and THP-1 cells after incubation with the test item ST-160.3 and ST-160.4 through the MTT assay.

The cells were incubated with the Test Items (ST-160.3 and ST-160.4; 1-30 μg/mL) for 24 hours, and then the cell viability assay was performed through the MTT method. An essay was made in duplicate. (A) ST-160.3 and em (B) ST-160.4. The viability percentage was calculated in relation to the Vehicle group (DMEM or RPMI1640 culture medium with 1% DMSO).

1-3. (canceled)

4. A tetracyclic triterpene 3-ols derivative with protein kinases or PKC isoforms that are therapeutically active against a condition selected from the group consisting of cancer, inflammation, and pain.

5. A method of treatment of a condition selected from the group consisting of consisting of cancer, inflammation, and pain comprising administering an effective amount of the tetracyclic triterpene 3-ols derivative of claim 4 to a patient in need thereof.

6. A pharmaceutical composition comprising the tetracyclic triterpene 3-ols derivative of claim 4 and a pharmaceutically accepted salt thereof or derivative, formulation, or cyclodextrin complex, or encapsulated form in bio-degradable polymers matrices.

7. A method of treatment of a condition selected from the group consisting of consisting of cancer, inflammation, and pain comprising administering an effective amount of the pharmaceutical composition of claim 6 to a patient in need thereof.

8. The tetracyclic triterpene 3-ols derivative of claim 1 further comprising a substance with a general formula presented in FIG. 1, wherein R can be sulfate, phosphate or phosphate ester, amino acid or peptide, PEG polymer or other hydrophilic polymer, dicarboxylic acids or its esters, and wherein R1-8 can be a moiety selected from the group consisting of hydrogen, methyl, hydroxyl, ether, hydroxymethyl and its ester or ether, and carboxyl groups and esters thereof.

9. A method of treatment of a condition selected from the group consisting of consisting of cancer, inflammation, and pain comprising administering an effective amount of the tetracyclic triterpene 3-ols derivative of claim 8 to a patient in need thereof.
